(19) United States
(12) Patent Application Publication
(10) Pub. No.: US 2006/0094909 A1
(43) Pub. Date: May 4, 2006

(54) Process for production of an alcohol

(75) Inventor: Alistair Chalmers Ramsay Brown, County Durham (GB)

Correspondence Address: RATNERPRESTIA, P O BOX 980, VALLEY FORGE, PA, 19482-0980, US

(73) Assignee: Johnson Matthey Public Limited Company, London (GB)

(21) Appl. No.: 10/528,289

(22) PCT Filed: Aug. 26, 2003
(86) PCT No.: PCT/GB03/03688

§ 371 Date: Sep. 8, 2005

(30) Foreign Application Priority Data
Sep. 17, 2002 (GB) 0221489.8

(51) Int. Cl. C07C 29/16

(52) U.S. Cl. 568/883

(57) ABSTRACT

A process for the production of alcohol, by hydrogenation of an aldehyde over a copper and zinc-containing catalyst includes the step of treating the reduced catalyst with a sulphur compound. The process reduces the hydrogenation of olefin contained in the aldehyde feed compared with a process using an untreated catalyst.

The present invention concerns selective hydrogenation processes and catalysts for use in such processes. In particular the invention concerns selective hydrogenation of aldehyde groups to alcohols in the presence of compounds containing olefin groups.

In the well-known processes for the production of alcohols by the so-called oxo process, an olefin is converted to an aldehyde by hydroformylation with carbon monoxide and hydrogen in the presence of a catalyst, which is typically a cobalt or a rhodium-based catalyst. The aldehyde is then hydrogenated over a copper-containing catalyst to give an alcohol which is purified by distillation. This process is often described as a two-stage oxo process because hydroformylation and hydrogenation are carried out in separate process stages. The hydroformylation product typically contains some unreacted olefin which may be removed from the process stream, normally by distillation, either before the hydrogenation or afterwards. The unreacted olefin may be returned to the hydroformylation stage either as a continuous recycle, or, more commonly and efficiently, as a separate feed stream on a campaign basis, i.e. as a discontinuous recycle. The oxo-process is described in various texts, for example in the Kirk-Othmer Encyclopaedia of Chemical Technology (John Wiley) 4^{th} Edn (1991), Vol1, p. 903-908.

The reaction product from the hydroformylation step contains typically 80% aldehyde, the remainder being mainly unconverted olefin. When the unreacted olefin is not separated until after the hydrogenation of the aldehyde hydroformylation product, it is possible for the olefin to be hydrogenated to saturated paraffin products, which are of relatively low value. It is economically desirable to minimise the hydrogenation of olefin so that it can be separated and recycled to hydroformylation for conversion to additional aldehyde. For this reason it is preferred to use a hydrogenation catalyst which is selective for the hydrogenation of the aldehyde group but which does not catalyse to a significant extent the hydrogenation of the C═C double bond in the olefin materials. Catalysts based on copper with chromium and/or zinc have been described for this process, as for example in U.S. Pat. No. 2,549,416 in which the use of such catalysts is described in detail.

U.S. Pat. No. 4,052,467 describes a process for the reduction of oxo-aldehydes to alcohols over a Cu/ZnO containing catalyst in which the feed contains a high concentration of thiophene (e.g. 500-2000 ppm) or another ring-type sulphur compound. The reaction conditions are specified to be in the range of temperature 450-550 degrees F. and a pressure of 800-1200 psig with a LHSV in the range 1.0-1.5 to ensure that the ring-type sulphur compounds in the feed do not decompose to sulphur or sulphur compounds which deleteriously affect the life of the catalyst. According to the invention, we provide a process for the production of an alcohol by the hydrogenation of a feed stream containing at least one aliphatic aldehyde compound and an olefin over a catalyst comprising a copper compound and a zinc compound, and optionally a catalyst support, comprising the step of treating said catalyst with an organic sulphur compound.

According to a second aspect of the invention, we provide a process for the production of an alcohol, comprising the steps of:
(a) reacting an olefin feed with hydrogen and carbon monoxide in a hydroformylation reactor in the presence of a suitable hydroformylation catalyst to form a hydroformylation product stream comprising an aldehyde and unreacted olefin,
(b) optionally treating said hydroformylation product stream to separate the catalyst from the remainder of the hydroformylation product stream,
(c) vaporising said hydroformylation product stream and feeding the vapour together with a source of hydrogen to a hydrogenation reactor containing a bed of a solid hydrogenation catalyst comprising a copper compound and a zinc compound, to form a hydrogenation product stream comprising an alcohol and unreacted olefin,
(d) separating said hydrogenation product stream into at least an alcohols stream and a stream containing said unreacted olefin,
   characterised in that the hydrogenation catalyst is treated with an organic sulphur compound before or during step (c).

We have found that the conversion of olefin to paraffin can be significantly reduced, without significantly changing the conversion of aldehyde to alcohol, by treating the hydrogenation catalyst with an organic sulphur compound. The unreacted olefin is normally recycled to the hydroformylation step either directly or, more preferably as a separate feed stream. If fed as a discrete recycle stream, the olefin which is separated from the hydrogenation product stream is preferably stored and then fed to the hydroformylation reactor as a distinct campaign so that the process conditions in the hydroformylation reactor may be optimised for the olefins contained in the recycle stream. We have also found that the conversion of aldehyde and product alcohol to alkane species in the hydrogenation reaction, e.g. by hydrogenolysis of the alcohol, can be significantly reduced using the process of the present invention, i.e. the selectivity of the catalyst to alcohol production is increased.

The treatment of the catalyst with an organic sulphur compound may be done either prior to or during the feeding of the aldehyde-containing feed stream to the catalyst. The sulphur compound can be added to the olefin feed before carrying out the hydroformylation in which case it is essential that the sulphur compound does not react under the conditions of hydroformylation, in particular it must not be a poison for the hydroformylation catalyst. Alternatively and preferably, the sulphur compound may be added to the crude aldehyde before it is fed to the hydrogenation reactor. It is preferred to treat the catalyst on-line, i.e. in situ in the hydrogenation reactor during the reduction of the catalyst with hydrogen or after the catalyst has been reduced.

Thiophene is an example of a suitable compound as it has a high sulphur content and it is an easily handled liquid. The sulphur compound is preferably fed to the catalyst as a vapour, optionally in the presence of a diluent which may be an inert compound (e.g. nitrogen), hydrogen or the aldehyde containing feed stream itself. Therefore preferred sulphur compounds are vaporisable under suitable conditions, preferably under the conditions at which the hydrogenation reaction is carried out. Other thiophene compounds such as benzothiophene or a thiol (a mercaptan), e.g. alkyl thiols such as 1-butanethiol, may also be suitable. It is preferred to use a sulphur compound which is conveniently stored and supplied on the hydrogenation plant. Thiophene, being a liquid under ambient conditions, is therefore particularly convenient to use in the process of the invention. Other sulphur compounds such as hydrogen sulphide may, however, be effective.

The sulphur treatment is preferably carried out on a freshly reduced catalyst by adding the sulphur compound to the hydrogenation feed over the first 1 to 10 days on line at a concentration equivalent to less than about 150 ppm by weight S, e.g. 5-150 ppm, (preferably less than 100 ppm S, e.g. from 5 to 100 ppm) based on feed. The quantity of sulphur compound required is equivalent to 0.2 to 10 kg of sulphur per tonne of catalyst, preferably from 1 to 5 kg of sulphur per tonne of catalyst. In practice it is desirable to adjust the amount of sulphur compound added by analysing the product stream. Therefore in preferred operation, the sulphur compound is added to the feed stream and the composition of the product stream is monitored to determine that the olefin hydrogenation has reduced to a suitable level. If, after the feed of sulphur compound has ceased, the olefin hydrogenation has increased then it may be desirable to treat the catalyst with a further quantity of the sulphur compound. Different feeds, catalyst types and operating conditions may affect the amount of sulphur compound required and, in practice, the skilled person will optimise the treatment to suit the conditions and materials in use. If too much sulphur is added, the catalyst activity may decline resulting in an increase in unconverted aldehyde.

The aldehyde and olefin are typically the products of a hydroformylation reaction operated as part of a production process for oxo-alcohols. Thus the feeds may vary widely from C3 to C20 olefins and above and their corresponding Cn+1 aldehydes. Normally the olefin is a C3 to about C15 compound or mixture of compounds and the aldehyde is C4 to about C16.

The olefin feeds may have terminal or internal unsaturation and may be linear or branched. Often, the olefin feed is a mixture of isomeric compounds produced as a fraction from a processed petroleum stream, for example a mixed heptene or nonene fraction. Another commercially useful feed is made by dimerising a lower olefin feed, e.g. an octene feed produced by dimerisation of a C4 olefin stream. Some oxo-processes incorporate an aldol condensation step in order to double the chain length of the product aldehyde, for example to make 2-ethylhexenal from a propylene feed, so the feed composition suitable for the invention incorporates such mixtures of olefin and aldehyde condensation product. An example of a suitable olefin is a C9 (nonenes) mixture. This is converted by hydroformylation into a C10 aldehyde which is hydrogenated to give the corresponding C10 alcohol (isodecanol).

The hydrogenation catalyst is normally supplied as pellets containing a reducible form of a copper compound such as copper oxide and zinc oxide. The catalyst may, optionally, also include a support material and/or promoter compounds. The catalyst support, if present is commonly alumina although other suitable catalyst supports may be used. Various promoters have been proposed for copper oxide/zinc oxide catalysts and these include alkali metal compounds, especially potassium or sodium, alkaline earth metals, e.g. magnesium, transition metal compounds, e.g. manganese, molybdenum, vanadium or zirconium or other metals such as cerium. Normally the promoter metal is present in the form of an oxide. Such promoters are known in the art. The copper compound is at least partially reduced to copper before the catalyst is brought on line. Suitable such catalysts are well known in the art and typically contain CuO and ZnO at weight ratios of from about 4:1 to 1:4, especially 2:1 to 1:3 (CuO:ZnO). As examples, suitable commercially available catalysts include PRICAT™ CZ 29/2, and PRICAT™ CZ 40/18, both available from Johnson Matthey Catalysts.

The conditions for the hydrogenation reaction may vary between about 20 barg and about 400 bar g. Typical aldehyde hydrogenation conditions are about 230 bar pressure and from 240 to 280° C., more preferably from about 245 to 270° C. at a liquid feed rate of 1.25 m³/hour of aldehyde per te of catalyst and a hydrogen rate of 3,000 to 10,000 m³/h of hydrogen per m³/hour of liquid (the hydrogen volume is that measured at 20° C. and atmospheric pressure).

If no treatment is carried out, a freshly reduced catalyst may convert up to 80% of the olefin in the crude aldehyde to paraffin. After treatment with a sulphur compound, the olefin conversion may be reduced to less than 30%, and often to less than 10%, so that more olefin is available to be recycled or otherwise recovered, leading to more favourable process economics.

The following examples are for illustration only.

### Example 1

A charge (16 tonne) of PRICAT™ CZ 29/2 CuO/ZnO catalyst available from Johnson Matthey Catalysts, was reduced to the active form by treatment with a low concentration of hydrogen in nitrogen, initially at 190° C. and a pressure of 50 barg increasing to 260° C. and 100 barg as the reduction neared completion.

A mixed C9 olefin (nonenes) stream was hydroformylated to give a mixture containing C10 aldehyde, unconverted nonenes and by-products including nonanes and high boiling C20 compounds. The crude aldehyde was hydrogenated to give a crude C10 alcohol (isodecanol) by passing it over the reduced hydrogenation catalyst in the presence of hydrogen at 250-270° C. and 235 barg. The liquid feed rate was 20 m³/hour and the hydrogen rate was 90 000 m³/hour, the rate for the latter being expressed as at 20° C. and atmospheric pressure.

Over the first six days on line a total of about 67 litres (15 gallons) of thiophene was added to the hydrogenation feed, equivalent to 1.7 kg S per tonne of catalyst. Over the next 10 days the olefin converted to paraffin was equivalent to 25% of the olefin in the feed. The residual aldehyde content of the crude alcohol product was less than 0.5% by weight.

### COMPARATIVE EXAMPLE 1A

A similar charge of catalyst, which was not treated with thiophene, gave 60-80% conversion of olefin to paraffin after 6 to 16 days on line. The residual aldehyde content of the crude alcohol was again less than 0.5% by weight.

### EXAMPLE 2

A laboratory scale hydrogenation of nonanal, which had been produced by the hydroformylation of di-isobutene, was carried out using a microreactor containing a 10 ml sample of PRICAT CZ 29/2 (35 wt % copper oxide/65 wt % zinc oxide) catalyst. Nonanal consists predominantly (about 90% by weight) of a single isomer, which is 3,5,5-trimethylhexanal.

The catalyst was reduced in the micro reactor at atmospheric pressure and 250° C. In a hydrogen-containing nitrogen stream flowing at 1 l/hr. the concentration of hydrogen in the gas stream was increased from 5% to 100% over a period of about 10 hours. The reactor was then brought up to operating pressure (200 barg) using neat hydrogen. The reactor was then operated at 300° C. at a gas:oil ratio of 7923:1, using the nonanal feed at a liquid feed rate of 15-20 ml/hour to show the performance of the catalyst before treatment with the sulphur compound. The gas:oil ratio is the hydrogen flow rate (\overline{R}m³/hr, i.e. measured at 20° C. and 1 atmosphere) divided by the liquid feed rate (m³/hr). 0.2 ml of thiophene was then added to the feed vessel contents (1.9 litres) and the reactor was run continuously under the same conditions until the feed had been consumed (7 days). Subsequently the reactor was run using aldehyde feed containing no thiophene. The feed and product streams were analysed using temperature-programmed capillary gas chromatography. Table 1 shows the concentration of the major components in the feed stream and in the product stream over the course of the reaction.

The abbreviations used in the table are:

244 tmp: 2,4,4-trimethyl pentane

224 tmh: 2,2,4-trimethyl hexane

aldehyde: 3,5,5-trimethylhexanal

alcohol: 3,5,5-trimethylhexanol

heavies: heavy end products including dimer (C18) ester, dimer alcohol, dimer ether, trimers and other high boiling by products.

TABLE 1

| Day | 244tmp | 224tmh | Aldehyde | Alcohol | Heavies |
|---|---|---|---|---|---|
| (Feed) | 2.4 | 0.1 | 85.4 | 8.5 | 0.58 |
| 0 | 3.3 | 16.2 | 0.1 | 75.9 | 4.66 |
| 1 | 3.6 | 9.1 | 0.1 | 81.7 | 7.25 |
| 2 | 3.2 | 2.7 | 0.2 | 89.8 | 6.91 |
| 3 | 3.4 | 1.8 | 0.4 | 89.8 | 6.85 |
| 4 | 3.0 | 1.7 | 0.4 | 90.2 | 7.07 |
| 5 | 2.6 | 2.3 | 0.3 | 90.1 | 7.5 |
| 6 | 2.9 | 2.6 | 0.3 | 89.9 | 6.92 |
| 7 | 2.5 | 2.6 | 0.3 | 90.1 | 7.21 |
| 8 | 2.7 | 2.7 | 0.3 | 89.9 | 7 |
| 9 | 2.2 | 2.3 | 0.3 | 92.3 | 4.38 |
| 10 | 2.7 | 2.5 | 0.3 | 91.8 | 3.78 |
| 11 | 2.9 | 2.7 | 0.3 | 90.3 | 5.03 |

There is very little unhydroformylated olefin in the crude aldehyde product used in this example. The concentration of 2,4,4-trimethyl pentane, which would be the product of hydrogenating di-isobutene is relatively low and constant. However, the concentration of 2,2,4-trimethyl hexane, which is at a very low level in the feed rises to over 16% when the hydrogenation takes place over un-treated catalyst. 2,2,4-trimethyl hexane is present as a product of the hydrogenolysis of the product alcohol. It is very noticeable that during and after treatment with thiophene, the concentration of 2,2,4-trimethyl hexane falls to about 2.5% with a corresponding increase in the concentration of desired product alcohol in the product stream.

1. A process for the production of an alcohol by the hydrogenation of an aliphatic aldehyde over a catalyst comprising a copper compound, and a zinc compound comprising the step of treating said catalyst with an organic sulphur compound.

2. A process according to claim 1, wherein said aliphatic aldehyde is present in a feed stream containing an olefin.

3. A process according to claim 2, wherein said feed stream is the product of a hydroformylation reaction.

4. A process according to claim 1, wherein said organic sulphur compound comprises thiophene.

5. A process according to claim 1 wherein said organic sulphur compound is present at a concentration of from 5 ppm to 150 ppm by weight of sulphur in a feed stream containing said aldehyde based upon the total mass of feed.

6. A process for the production of an alcohol, comprising the steps of:
(a) reducing a catalyst bed provided within a reactor with a hydrogen-containing gas stream;
(b) feeding to the bed of said reduced catalyst a gaseous feed stream comprising an aldehyde, a sulphur compound, and hydrogen for a period of time sufficient to provide from 0.2 to 10 kg of sulphur (S) per ton of catalyst wherein a concentration of said sulphur compound in said feed stream is less than 150 ppm; and
(c) subsequently feeding to said catalyst bed a feed stream containing no sulphur compound.

7. A process for the production of an alcohol, comprising the steps of:
(a) reacting an olefin feed with hydrogen and carbon monoxide in a hydroformylation reactor in the presence of a suitable hydroformylation catalyst to form a hydroformylation product stream comprising an aldehyde and unreacted olefin;
(b) vaporising said hydroformylation product stream and feeding the vapour together with a stream of a hydrogen-containing gas to a hydrogenation reactor containing a bed of a solid hydrogenation catalyst comprising a copper compound and a zinc compound, to form a hydrogenation product stream comprising at least an alcohol and said unreacted olefin; and
(c) separating said hydrogenation product stream into at least an alcohol stream and a stream containing said unreacted olefin,
wherein the hydrogenation catalyst is treated with an organic sulphur compound before or during step (b).

8. A process according to claim 1, wherein the catalyst further comprises at least one of a catalyst support and a promoter compound.

9. A process according to claim 7 further comprising the step of treating said hydroformylation product stream of step (a) to separate the catalyst from the remainder of the hydroformylation product stream.
